SEQUENCE LISTING (35 sequences)

SEQ ID NO: 1 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 2 - protein, 118 aa, Artificial Sequence [sequence omitted]

SEQ ID NO: 3 - protein, 113 aa, Artificial Sequence [sequence omitted]

SEQ ID NO: 4 - protein, 8 aa, Artificial Sequence
  synthetic
GFTFNNYA

SEQ ID NO: 5 - protein, 8 aa, Artificial Sequence
  synthetic
ISGSGGTT

SEQ ID NO: 6 - protein, 11 aa, Artificial Sequence
  synthetic
AKDSNWGNFDL

SEQ ID NO: 7 - protein, 12 aa, Artificial Sequence
  synthetic
QSVLYRSNNRNF

SEQ ID NO: 8 - protein, 3 aa, Artificial Sequence
  synthetic
WAS

SEQ ID NO: 9 - protein, 9 aa, Artificial Sequence
  synthetic
QQYYTTPYT

SEQ ID NO: 10 - protein, 662 aa, Homo sapiens
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 11 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 12 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 13 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 14 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 15 - protein, 11 aa, Artificial Sequence
  synthetic
DGTRFHRQASK

SEQ ID NO: 16 - protein, 11 aa, Artificial Sequence
  synthetic
SIPWNLERITP

SEQ ID NO: 17 - protein, 8 aa, Artificial Sequence
  synthetic
SIPWNLER

SEQ ID NO: 18 - protein, 11 aa, Artificial Sequence
  synthetic
SIPWNLERIIP

SEQ ID NO: 19 - protein, 8 aa, Artificial Sequence
  synthetic
AKDPWRLP

SEQ ID NO: 20 - protein, 9 aa, Artificial Sequence
  synthetic
AARRGYLTK

SEQ ID NO: 21 - protein, 8 aa, Artificial Sequence
  synthetic
SKEAWRLP

SEQ ID NO: 22 - protein, 9 aa, Artificial Sequence
  synthetic
AARRGYVIK

SEQ ID NO: 23 - protein, 5 aa, Artificial Sequence
  synthetic
SYYMH

SEQ ID NO: 24 - protein, 10 aa, Artificial Sequence
  synthetic
GYTFTSYYMH

SEQ ID NO: 25 - protein, 7 aa, Artificial Sequence
  synthetic
GYTFTSY

SEQ ID NO: 26 - protein, 16 aa, Artificial Sequence
  synthetic
EISPFGGRTNYNEKFK

SEQ ID NO: 27 - protein, 6 aa, Artificial Sequence
  synthetic
SPFGGR

SEQ ID NO: 28 - protein, 9 aa, Artificial Sequence
  synthetic
ERPLYASDL

SEQ ID NO: 29 - protein, 11 aa, Artificial Sequence
  synthetic
RASQGISSALA

SEQ ID NO: 30 - protein, 7 aa, Artificial Sequence
  synthetic
SASYRYT

SEQ ID NO: 31 - protein, 9 aa, Artificial Sequence
  synthetic
QQRYSLWRT

SEQ ID NO: 32 - protein, 118 aa, Artificial Sequence
  synthetic
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 33 - protein, 107 aa, Artificial Sequence
  synthetic
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 34 - protein, 214 aa, Artificial Sequence
  synthetic
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 444 aa, Artificial Sequence
  synthetic
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2017/0056504 A1
(43) Pub. Date: Mar. 2, 2017

(54) METHOD OF EXTENDING HALF-LIFE OF CRYSTALINE ANTIBODIES

(71) Applicants: Kenneth I. Kohn, West Bloomfield, MI (US); Toshihisa Kawai, Brookline, MA (US)

(72) Inventors: Kenneth I. Kohn, West Bloomfield, MI (US); Toshihisa Kawai, Brookline, MA (US)

(21) Appl. No.: 15/254,316

(22) Filed: Sep. 1, 2016

Related U.S. Application Data
(60) Provisional application No. 62/213,311, filed on Sep. 2, 2015.

(51) Int. Cl. A61K 47/26; A61K 9/00; C07K 16/40

(52) U.S. Cl. CPC A61K 47/26; C07K 16/40; A61K 9/0053; C07K 2317/24; A61K 2039/505

(57) ABSTRACT

A composition for extending the half-life of a crystalline antibody, including synergistically effective amounts of a half-life-extending compound and a crystalline antibody. A composition for extending the half-life of a crystalline antibody including synergistically effective amounts of lactulose and a crystalline anti-PCSK9 antibody. A method of extending the half-life of a crystalline antibody, by administering a synergistically effective amount of a half-life-extending compound and a crystalline antibody to an individual, and extending the half-life of the crystalline antibody. A method of promoting antibody recycling by affecting a receptor for an antibody to recycle the antibody.

### BACKGROUND OF THE INVENTION

1. Technical Field

The present invention relates to compounds in combination with crystalline antibodies. More specifically, the present invention relates to methods of extending the half-life of crystalline antibodies.

2. Background Art

Antibodies are large Y-shaped proteins produced by the body's immune system after detection of antigens, i.e. any numerous foreign substances, including bacteria, fungi, parasites, viruses, and chemicals. Antibodies elicit the body's immune response to the antigens. An antibody has structure that is specific for an epitope on an antigen that allows the antibody to bind with the antigen. The binding can neutralize the antigen or tag it for destruction by the body.

Antibodies are also used as therapeutic agents. Monoclonal antibodies (mAbs) can bind with specific cells or proteins in the body and induce the immune system to attack and destroy those cells or proteins. mAbs are remarkably versatile protein molecules with numerous applications in human health. More than 30 mAb drugs have been approved for treatment or prevention of diseases and approximately 400 mAbs are currently in clinical studies. The indications of these studies are diverse including autoimmune diseases, cancers, metabolic disorders, and infectious diseases.

For example, antibody therapy can be useful in treating cancer, as the immune system does not recognize tumor cells as invasive foreign substances since they are the body's own cells. For example, radioactively conjugated antibodies can be used to target lymphomas and deliver radiation to a specific site with tositumomab. Antibodies can be conjugated with drug-activating enzymes in prodrug therapy wherein the drug is activated at the cancer cells bound to the antibodies. Antibodies can further be conjugated to liposomes containing therapeutics and delivered to cancer cells.

Antibody therapy is also useful in treating autoimmune diseases, such as rheumatoid arthritis, Crohn's disease, ulcerative colitis, transplant rejection, and asthma. Infliximab (REMICADE®, Janssen Biotech, Inc) binds TNF-α and prevents its binding to receptors to induce inflammatory response, and is indicated for psoriasis, Crohn's disease, ankylosing spondylitis, psoriatic arthritis, rheumatoid arthritis, and ulcerative colitis. Adalimumab (HUMIRA®, AbbVie) also binds to TNF-α and is indicated for rheumatoid arthritis, psoriatic arthritis, ankylosing spondylitis, Crohn's disease, ulcerative colitis, moderate to severe chronic psoriasis, and juvenile idiopathic arthritis. Basiliximab (SIMULECT®, Novartis) binds to the alpha chain subunit of the IL2 receptor of T cells and competes with IL-2 to prevent an immune response, and is indicated for organ transplant rejection prevention. Omalizumab (XOLAIR®, Genentech/Novartis) binds to free human immunoglobulin E (IgE) and is indicated for treating severe to moderate allergic asthma.

The high specificity to target molecule along with less possibility of causing side effect are advantages of monoclonal antibody drugs over the conventional small molecule drugs. Furthermore, another merit of monoclonal antibody drugs is derived from its longer serum half-life (7-30 days) compared to that of small molecule drugs (6-24 hours). The half-life of an antibody is the time it takes for half of the administered antibodies to be eliminated from the body. Different antibodies have different half-lives. Murine antibodies have short half-lives when administered in humans. Chimeric (human and murine fused) and humanized antibodies have improved serum half-lives but can have decreased affinity for antigens.

In 2014, seven out of top ten best-selling drugs in the US were monoclonal antibody drugs, suggesting that monoclonal antibody drugs are replacing the market share of small molecule drugs. In spite of above noted advantages of mAb drugs, intravenous injection which is required for the administration of most of mAb drugs is considered to be the drawback of mAb drugs. Therefore, mAb drugs that possesses longer serum half-life or the method increasing the serum half-life of mAb drug is profoundly sought in the course of R&D.

Among a number of monoclonal antibody drugs that are currently under clinical trial, anti-PCSK9 mAb drugs developed for reduction of low-density lipoprotein (LDL) cholesterol in blood are beginning Phase III clinical trials to assess their safety and efficacy in humans, and to determine if they can improve outcomes in cardio vascular diseases. PCSK9 is a serine protease that reduces both hepatic and extrahepatic low-density lipoprotein (LDL) receptor levels which, in turn, increase plasma LDL cholesterol, based on the mechanism that LDL receptor functions to bind LDL in plasma and internalizes into cytoplasm where LDL is degraded by ubiquitin ligase. Serum half-life of anti-PCSK9 mAb are remarkably shorter than the other mAb drugs, represented by a range between 2-7 days shown in preclinical animal models (Poirier and Mayer, 2013) probably due to the mechanism that immune complex formed between anti-PCSK9 mAb and PCSK9 in the serum is filtered at liver which is, in turn, discharged into intestine through bile duct. Based on such a short serum half-life of anti-PCSK9 mAb, the progresses on some of clinical trials appears to be stranded, due to the high frequency of invasive intra venous injection of anti-PCSK9 mAb to the patients.

In all of the above uses of antibodies, they are delivered via i.v. infusion to the patient because the antibodies are prone to aggregation and are unable to be delivered through thin needles at high concentrations. It is therefore desirable to provide a low volume suspension with a crystalline formation of antibodies to provide other routes of administration besides iv infusion. For example, insulin has been successfully crystallized to provide stable and long acting drugs. However, insulin also is easy to crystallize by contacting with zinc ions. Antibodies are considerably more difficult to crystallize, due to their large size, flexibility of their structure, and the presence of surface oligosaccharides.

It has previously been shown that abnormal immunoglobulin (Ig) crystals can be formed in 1848, and spontaneous crystallization of another abnormal Ig was detected in 1938. Further studies showed that crystalline Igs retain their immunological activities when redissolved (Nisonoff, et al. 1968). Early studies were unsuccessful at producing large amounts of crystals necessary for human administration, because of the use of vapor diffusion as well as the use of agents unacceptable for biocompatibility with humans.

Yang, et al. (PNAS 2003) were the first to explore crystalline antibodies for therapeutic use and showed that batch crystallization was successful in producing crystalline rituximab, trastuzumab, and infliximab. High-concentration, low-viscosity crystalline preparations were produced. Administration by subcutaneous injection of each crystalline preparation provided a long pharmacokinetic serum profile and dose-dependent inhibition of tumor growth in mice. It was found that crystalline mAbs took longer to enter the bloodstream, provided a longer calculated maximum concentration (Cₘₐₓ) value, and a significantly longer half-life compared with similar s.c. or i.v. injections of non-crystalline mAbs. The crystalline mAbs also provided high bioavailability.

U.S. Pat. No. 7,833,525 to Shenoy, et al. discloses crystals of whole antibodies and fragments thereof, and formulations and compositions comprising such crystals. More particularly, methods are provided for the crystallization of high concentrations of whole antibodies, and fragments thereof, in large batches, and for the preparation of stabilized whole antibody crystals for use alone, or in dry or slurry formulations or compositions. Also disclosed are methods for stabilization, storage and delivery of biologically active whole antibody crystals. Shenoy, et al. discloses crystallization of, and use of crystals of, all of the immunoglobulin classes IgG, IgM, IgA, IgD, IgE, and serum IgA (sIgA) as well as the subclasses IgG1, IgG2, IgG3 and IgG4, IgM1 and IgM2, and IgA1 and IgA2, as well as scFv fragments and Fab antibody fragments, from all the immunoglobulin classes and subclasses.

U.S. Pat. No. 8,404,819 to Borhani, et al. discloses batch crystallization methods for crystallizing an anti-hIL-12 antibody that allows the production of the antibody on an industrial scale, antibody crystals obtained according to the methods, compositions containing the crystals, and methods of using the crystals and the compositions. Borhani, et al. also disclose embedding crystals with encapsulation to provide controlled or sustained release.

U.S. Pat. No. 8,436,149 to Borhani, et al. discloses batch crystallization method for crystallizing an anti-hTNFalpha antibody which allows the production of said antibody on an industrial scale; antibody crystals as obtained according to said method; compositions containing said crystals as well as methods of use of said crystals and compositions.

U.S. Patent Application Publication No. 2011/0020322 to Wilkins, et al. discloses although antibodies, especially full-length antibodies, are traditionally difficult to crystallize, CD20 antibodies can be successfully crystallized from Harvested Cell Culture Fluid (HCCF) of mammalian cell cultures. In particular, the identification of conditions that allow the formation of CD20 antibody crystals, including large, uniform, CD20 antibody crystals, from HCCF is disclosed. Accordingly, a process for purifying CD20 antibodies from mammalian cell cultures, including a crystallization step in the purification scheme. Incorporation of a crystallization step in the CD20 antibody purification scheme eliminates chromatographic steps and their inherent limits of scalability while maintaining comparable yields to traditional purification schemes that use multiple chromatographic purification steps, without crystallization is disclosed. Implementing crystallization into the purification process results in marked time and cost savings, without compromising efficiency, product yields or product quality.

U.S. Patent Application Publication No. 2015/0147337 to Reichert, et al. discloses crystalline forms of antibodies to human IL-23, such as antibodies to human IL-23p19, as well as methods of producing such crystalline forms, and uses of such crystalline forms, e.g. in treatment of inflammatory, autoimmune, and proliferative disorders. In various embodiments, the anti-huIL-23 antibody crystals, such as anti-huIL-23p19 antibody crystals are obtainable by batch crystallization methods, vapor diffusion methods, liquid-liquid diffusion methods, and dialysis. In other aspects, suspensions of the crystalline anti-huIL-23 antibodies are disclosed, including those at higher concentrations and lower viscosities than would be possible with a corresponding non-crystalline solution at the same concentration of antibody. In other embodiments, the anti-huiL-23 antibody crystals have increased stability, i.e. they maintain biological activity of the anti-huiL-23 antibody, such as anti-huiL-23p 19 antibody, longer than corresponding solution formulations.

Lactulose is a synthetic, non-digestible sugar used in the treatment of chronic constipation and hepatic encephalopathy. Those medical potencies by lactulose are mediated by its effect to promote the activity of bacteria in the gut. It is reported that during the fermentation of lactulose, short-chain fatty acids are formed with consequent lowering of the colon pH and modification of the colon microflora (Sahota et al., 1982; Salminen and Salminen, 1997). Such pH lowering effects by lactulose is derived from its effect to promote the growth of lactic acid bacteria and bifidobacteria and, more specifically, Lactobacillus acidophilus in the colon (Salminen and Salminen, 1997). In the human clinical trial, chronic oral administration of lactulose (20 g/twice/day, P.O. for 8 days) remarkably lowered the cecum pH from 7.0 to the range between 5.0-6.0 (Florent et al., 1985). These studies clearly support that lactulose can lower the pH of colon by modulation the activity of microflora. It is noteworthy that majority of gut bacteria colonize in large intestine, and lesser in small intestine, prebiotic effects of lactulose can be attained in large intestine, but not in small intestine.

On the other hand, neonatal Fc receptor (FcRn) is a unique IgG fc receptor that can transpose IgG through epithelium of intestinal lumens (Yoshida et al., 2004). This receptor also plays a role in salvage of IgG in adults through its role in the process of endocytosis in endothelial cells. Fc receptors in the acidic endosomes bind to IgG internalized through pinocytosis, recycling it to the cell surface and releasing it at the basic pH of blood, and thereby prevent IgG from undergoing lysosomal degradation. This mechanism may provide an explanation for the greater half-life of IgG (about 1-3 weeks) in the blood compared to that of other isotypes, such as IgA and IgM. Very interestingly, it is reported that the immune complex composed of IgG antibody and target antigen can be dissociated inside of endosomes during FcRn-mediated intracellular trafficking (Devanaboyina et al., 2013), suggesting that mAb drugs that bind to target antigen can also be separated by FcRn-mediated IgG recycling processes. However, as noted above, the neutral pH (pH 7) of large intestine of human adult where FcRn is prominently expressed (Hornby et al., 2014) may not offer the appropriate condition for FcRn mediated IgG recycling.

There remains a need to extend the half-lives of crystalline antibodies in order to extend their therapeutic effect in the body.

### SUMMARY OF THE INVENTION

The present invention provides for a composition for extending the half-life of a crystalline antibody, including synergistically effective amounts of a half-life-extending compound and a crystalline antibody.

The present invention provides for a composition for extending the half-life of a crystalline antibody including synergistically effective amounts of lactulose and a crystalline anti-PCSK9 antibody.

The present invention provides for a method of extending the half-life of a crystalline antibody, by administering a synergistically effective amount of a half-life-extending compound and a crystalline antibody to an individual, and extending the half-life of the crystalline antibody.

The present invention also provides for a method of promoting antibody recycling by affecting a receptor for an antibody to recycle the antibody.

### DESCRIPTION OF THE DRAWINGS

Other advantages of the present invention are readily appreciated as the same becomes better understood by reference to the following detailed description when considered in connection with the accompanying drawings wherein:

FIG. 1 is a depiction of an in vitro xPCSK9 mAb transportation assay;

FIG. 2 is a graph of transposed xPCSK9 mAb versus incubation time;

FIG. 3 is a depiction of an in vitro xPCSK9 mAb transportation assay;

FIG. 4 is a graph of concentration of xPCSK9 mAb;

FIG. 5A is depiction of in vivo examination of xPCSK9 mAb serum half life for oral administration with lactulose and FIG. 5B is a depiction for oral administration with control water;

FIG. 6A is a graph of amounts of xPCSK9 mAb detected in serum, FIG. 6B is a graph of calculated serum half-life, and FIG. 6C is a graph of pH in the large intestine;

FIG. 7 is a graph of LDL/VLDL in blood serum;

FIG. 8A is a is depiction of in vivo examination of xPCSK9 mAb serum half life for administration of xPCSK9 mAb into normal wild type mice and FIG. 5B is a depiction for administration of xPCSK9 mAb into FcRn-KO mice;

FIG. 9 is a graph of xPCSK9 mAb in serum;

FIG. 10A is depiction of in vivo examination of xPCSK9 mAb serum half life for oral administration with lactulose and FIG. 10B is a depiction for oral administration with lactulose and antibiotics;

FIG. 11A is a graph of amounts of xPCSK9 mAb detected in serum and FIG. 11B is a graph of pH in the large intestine; and

FIG. 12A is a graph of the amount of total xPCSK9-mAb (free and PCSK9-bound xPCSK9 mAb) in intestines, FIG. 12B is a graph of the amount of free xPCSK9 mAb in intestines, and FIG. 12C is a graph of the pH of the intestines.

### DETAILED DESCRIPTION OF THE INVENTION

The present invention provides generally for compositions and methods for extending the half-life of a crystalline antibody. The composition generally includes a half-life-extending compound in combination with a crystalline antibody. By using a crystalline antibody in combination with the half-life-extending compound, a traditionally injectable therapeutic can be transformed into an oral therapeutic and the half-life of the crystalline antibody can be increased. Furthermore, synergistic effects of the combination can include reduction and/or elimination of inflammation and positive effects on lipid metabolism.

“Antibody” as used herein, refers to a protein that binds with a specific antigen and can neutralize or signal destruction of the antigen. The antibodies can be murine, human, or chimeric. Preferably, the antibodies are crystalline.

“Crystalline”, “crystal”, or “crystallized” as used herein refers to an ordered form of the solid state of matter, which is distinct from a second form—the amorphous solid state, which exists essentially as an unorganized, heterogeneous solid. Crystals are regular three-dimensional arrays of atoms, ions, molecules (e.g., proteins such as antibodies), or molecular assemblies (e.g., antigen/antibody complexes). Crystals are lattice arrays of building blocks called asymmetric units (which consist of the substance to be crystallized) that are arranged according to well-defined symmetries into unit cells that are repeated in three-dimensions.

“Half-life” as used herein, refers to the time it takes for half of administered antibodies to be eliminated from the body.

“Half-life-extending compound” as used herein, refers to a compound that has the ability to extend the half-life of an antibody, and is further described below.

“Hydrogen” as used herein, refers to the composition H₂ (also written herein as “H2”), but can also include molecular hydrogen (H) and any composition capable of releasing hydrogen. In other words, H2 molecules per se can be administered, a prodrug able to release H2, or a compound that can cause the release of H2 within the body can be administered, as further described below.

The half-life-extending compound is preferably one of the following types of compounds: a non-absorbable sugar, a compound that is able to convert NH3 to NH4+ in the body, a prebiotic, or a hydrogen-generating compound. It should be understood that the half-life-extending compound can have one of these properties, several, or all of these properties, depending upon the specific compound. Preferably, the half-life-extending compound also has the ability to reduce and/or eliminate inflammation.

The half-life-extending compound is preferably lactulose, or homologues thereof. Lactulose has all of the properties described above (i.e. it is a non-absorbable sugar, a compound that is able to convert NH3 to NH4+ in the body, a prebiotic, a hydrogen-generating compound, and reduces/eliminates inflammation). Lactulose is a synthetic sugar that is a disaccharide of a molecule of fructose and a molecule of galactose. It is currently indicated for constipation treatment or for hepatic encephalopathy in removing ammonia from blood. Lactulose is non-absorbable and fermented by intestinal bacteria, resulting in the production of hydrogen. A lactulose hydrogen breath test has previously been used to detect irritable bowel syndrome by detecting an abnormal amount of hydrogen in the breath. However, the creation of hydrogen in the present invention is a positive effect of the compound so that inflammation can be treated. Lactulose can be administered in doses from 40 mL to over 1000 mL per day; however, due to the synergism with the crystalline antibody, a lower dose can be preferred. For example, lactulose can be administered in an amount lower than indicated for treating constipation of less than 10 g/day.

When the half-life-extending compound is a non-absorbable sugar, it can also be any other monosaccharide, polysaccharide, or other non-saccharide sweetener, such as, but not limited to, glucose, galactose, fructose, mannitol, inulin, sucralose, aspartame, dextrose, maltodextrin, homologues, or combinations thereof. Inulin in particular is composed of a heterogeneous collection of fructose polymers, is not absorbed in the blood, and elevates the amount of H2 in mice with oral administration. The non-absorbable sugar can be administered in amounts similar to lactulose.

When the half-life-extending compound is a compound that is able to convert NH3 to NH4+ in the body, i.e. upregulate levels of ammonium, it can be any of the sugars listed above, or any other suitable compound that is able to convert NH3 to NH4+. Ammonium (NH4+) is generally produced when ammonia (NH3) reacts with proton donors (compounds that can donate a hydrogen ion). Therefore, any compound that can donate a proton can be used to convert NH3 to NH4+. Most preferably, lactulose is used to convert NH3 to NH4+.

The half-life-extending compound can also be any suitable prebiotic, including, but not limited to, lactulose (and other disaccharides), inulin, fructooligosaccharides (FOS) (produced by a degradation of inulin), xylooligosaccharides (XOS), polydextrose, oligofructose-enriched inulin, resistant starch, galactooligosaccharides (GOS), transgalactooligosaccharide, mannooligosaccharides, or tagatose (and other monosaccharides). The prebiotics can be short-chain (containing 2-8 links per saccharide molecule and typically fermenting more quickly in the right side of the colon), long-chain (containing 9-64 links per saccharide molecule and fermenting slower in the left side of the colon), or full-spectrum (containing 2-64 links per saccharide molecule and fermenting through the whole colon). The prebiotics can stimulate the growth of many types of bacterial, but especially beneficial bifidobacteria and lactic acid species in the gut. Prebiotics are generally dosed at 4-15 g, but due to synergy with other compounds described herein, lower doses can be used.

The half-life-extending compound can be a hydrogen-generating compound that acts to generate an increase of hydrogen in the body by several different methods, such as, but not limited to, releasing hydrogen in the body, or inducing production of hydrogen in the body. The hydrogen-generating compound can also alter lipid metabolism and/or reduce or eliminate inflammation. The generation of hydrogen can occur anywhere in the body as desired, and can be tailored to occur in a specific site. The hydrogen-generating compound can be H2 molecules, or a composition that includes a releasable H2 moiety. Alternatively, the hydrogen-generating compound can be a compound that induces hydrogen to be released by the body itself or by another compound in the body. For example, the hydrogen-generating compound can be any compound that induces bacteria in the stomach to release or produce H2. The hydrogen-generating compound can also be a H2 infused liquid that can be a drink or administered by intravenous infusion, or any other method described herein. Combinations of any of the methods of generating hydrogen in the body can also be used.

Most preferably, the crystalline antibody is a lipid-lowering crystalline antibody, i.e. any crystalline antibody that has the ability to effect cholesterol metabolism to reduce high cholesterol in an individual.

The lipid-lowering crystalline antibody can be the monoclonal antibody (MAb) anti-PCSK9 in crystalline form. The crystalline anti-PCSK9 MAb can be any suitable antibody that binds with PCSK9, or a portion(s) thereof, in order to block its mechanism of action, but can also preferably be any specific anti-PCSK9 described below. Preferably, the crystalline anti-PCSK9 MAb is injected intravenously, but can be administered in any method described herein.

Proprotein convertase subtilisin/kexin type 9 (PCSK9) plays a critical role in cholesterol metabolism by controlling the levels of LDL particles that circulate in the bloodstream. PCSK9 increases plasma LDL cholesterol by promoting degradation of the LDL receptor, which mediates LDL endocytosis in the liver, the major route of LDL clearance from circulation. Anti-PCSK9 MAb blocks PCSK9 so that LDL levels are reduced. The ammonium ion (NH4+) can protect PCSK9-mediated LDL-receptor (LDLR) degradation. The lipid metabolism-altering compound, especially lactulose described below, is able to convert toxic ammonia (NH3) derived from gut bacteria to ammonium ion (NH4+), both in the gut as well as systemically throughout the body. Elevated ammonium ion maintains the good LDLR that captures and destroys bad LDL in the liver. Therefore, the combination of the lipid metabolism-altering compound and crystalline anti-PCSK9 MAb is synergistic and provides additive effects in lowering high cholesterol. Further synergy can exist when the lipid metabolism-altering compound, lipid-lowering crystalline antibody (crystalline anti-PCSK9 MAb), and statins are used in combination.

U.S. Pat. No. 8,062,640 to Sleeman, et al. discloses a human antibody or antigen-binding fragment of a human antibody that specifically binds and inhibits human proprotein convertase subtilisin/kexin type 9 (hPCSK9) characterized by the ability to reduce serum LDL cholesterol by 40-80% over a 24, 60 or 90 day period relative to predose levels, with little or no reduction in serum HDL cholesterol and/or with little or no measurable effect on liver function, as determined by ALT and AST measurements.

The crystalline anti-hPCSK9 antibody or antigen-binding fragment of an antibody can bind an epitope within the catalytic domain of hPCSK9, which is about 153 to 425 of SEQ ID NO: 755 (SEQ ID NO: 1 herein); more specifically, an epitope from about 153 to about 250 or from about 250 to about 425; more specifically, the antibody or antibody fragment of the invention binds an epitope within the fragment from about 153 to about 208, from about 200 to about 260, from about 250 to about 300, from about 275 to about 325, from about 300 to about 360, from about 350 to about 400, and/or from about 375 to about 425.

The crystalline anti-hPCSK9 antibody or antigen-binding fragment of a crystalline antibody can bind an epitope within the propeptide domain (residues 31 to 152 of SEQ ID NO: 755 (SEQ ID NO: 1 herein)); more specifically, an epitope from about residue 31 to about residue 90 or from about residue 90 to about residue 152; more specifically, the antibody or antibody fragment binds an epitope within the fragment from about residue 31 to about residue 60, from about residue 60 to about residue 90, from about residue 85 to about residue 110, from about residue 100 to about residue 130, from about residue 125 to about residue 150, from about residue 135 to about residue 152, and/or from about residue 140 to about residue 152.

The crystalline anti-hPCSK9 antibody or antigen-binding fragment of a crystalline antibody can bind an epitope within the C-terminal domain, (residues 426 to 692 of SEQ ID NO: 755 (SEQ ID NO: 1 herein)); more specifically, an epitope from about residue 426 to about residue 570 or from about residue 570 to about residue 692; more specifically, the antibody or antibody fragment binds an epitope within the fragment from about residue 450 to about residue 500, from about residue 500 to about residue 550, from about residue 550 to about residue 600, and/or from about residue 600 to about residue 692.

The crystalline antibody or antibody fragment can bind an epitope that includes more than one of the enumerated epitopes within the catalytic, propeptide or C-terminal domain, and/or within two or three different domains (for example, epitopes within the catalytic and C-terminal domains, or within the propeptide and catalytic domains, or within the propeptide, catalytic and C-terminal domains.

The crystalline antibody or antigen-binding fragment can bind an epitope on hPCSK9 comprising amino acid residue 238 of hPCSK9 (SEQ ID NO: 755 (SEQ ID NO: 1 herein)).

The crystalline mAbs can be full-length (e.g., an IgG1 or IgG4 antibody) or may comprise only an antigen-binding portion (e.g., a Fab, F(ab′).sub.2 or scFv fragment), and may be modified to affect functionality, e.g., to eliminate residual effector functions (Reddy et al. (2000) J. Immunol. 164:1925-1933).

The crystalline anti-PCSK9 MAb can include heavy and light chain CDR sequences from the HCVR (heavy chain variable region) and LCVR (light chain variable region) sequence pair having SEQ ID NOs: 90/92 (SEQ ID NOs: 2 and 3 herein). The crystalline anti-PCSK9 MAb can include heavy and light chain CDR sequences having SEQ. ID NOs; 76, 78, 80, 84, 86, and 88 (SEQ ID NOs: 4, 5, 6, 7, 8, and 9 herein). The crystalline anti-PCSK9 MAb can include an HCVR having the amino acid sequence, of SEQ ID NO:90 (SEQ ID NO: 2 herein) and an LCVR having the amino acid sequence of SEQ ID NO: 92 (SEQ ID NO: 3 herein).

U.S. Pat. Nos. 8,030,457, 8,168,762, U.S. Patent Application Publication Nos. 2011/0027287, 2012/0020975, 2012/0027765, 2012/0213797, and 2012/0251544 to Jackson, et al. disclose antigen binding proteins that interact with PCSK9 to decrease the LDLR lowering effect of PCSK9 on LDLR, the PCSK9 preferably having the amino acid sequence of SEQ ID NO: 1 (SEQ ID NO: 10 herein), or variants or mutations thereof.

An isolated neutralizing antigen binding protein or human monoclonal antibody that binds to a PCSK9 protein including the amino acid sequence of SEQ ID NO: 1 (SEQ ID NO: 10 herein) as described in U.S. Pat. No. 8,030,457, wherein the neutralizing antigen binding protein includes: a heavy chain polypeptide including the following complementarity determining regions (CDRs): a heavy chain CDR1 that is a CDR1 in SEQ ID NO: 49 (SEQ ID NO: 11 herein); a heavy chain CDR2 that is a CDR2 in SEQ ID NO: 49 (SEQ ID NO: 11 herein); a heavy chain CDR3 that is a CDR3 in SEQ ID NO: 49 (SEQ ID NO: 11 herein) and a light chain polypeptide including the following CDRs: a light chain CDR1 that is a CDR1 in SEQ ID NO: 23 (SEQ ID NO: 12 herein); a light chain CDR2 that a CDR2 in SEQ ID NO: 23 (SEQ ID NO: 12 herein); and a light chain CDR3 that is a CDR3 in SEQ ID NO: 23 (SEQ ID NO: 12 herein).

An isolated neutralizing antigen binding protein or human monoclonal antibody that binds to a PCSK9 protein of the amino acid sequence of SEQ ID NO: 1 (SEQ ID NO: 10 herein) can be used as described in U.S. Pat. No. 8,168,762, wherein the neutralizing antigen binding protein includes a heavy chain polypeptide including the following complementarity determining regions (CDRs): a heavy chain CDR1 that is a CDR1 in SEQ ID NO: 67 (SEQ ID NO: 13 herein); a heavy chain CDR2 that is a CDR2 in SEQ ID NO: 67 (SEQ ID NO: 13 herein); a heavy chain CDR3 that is a CDR3 in SEQ ID NO: 67 (SEQ ID NO: 13 herein); and a light chain polypeptide including the following CDRs: a light chain CDR1 that is a CDR1 in SEQ ID NO: 12 (SEQ ID NO: 14 herein); a light chain CDR2 that a CDR2 in SEQ ID NO: 12 (SEQ ID NO: 14 herein); and a light chain CDR3 that is a CDR3 in SEQ ID NO: 12 (SEQ ID NO: 14 herein).

WO2011/027257 to Champion, et al. discloses an immunogen of an antigenic PCSK9 peptide and optionally an immunogenic carrier. The antigenic PCSK9 peptide can be a portion of PCSK9 comprising between 4 to 20 amino acids and, when administered to a subject, is able to lower the LDL-cholesterol level in blood of said subject. Preferably, said subject is a mammal, preferably a human. Preferably, said antigenic PCSK9 peptide is able to lower the LDL-cholesterol level by at least 2%, 5%, 10%, 20%, 30% or 50%. The antigenic PCSK9 peptide can be a portion of PCSK9 which participates in the interaction of PCSK9 with the LDL receptor. Preferably, the antigenic PCSK9 peptide is a peptide of sequence SEQ ID NO: 56, 184, 187, 332, 445, 482, 525, or 563 (SEQ ID NOs: 15, 16, 17, 18, 19, 20, 21, and 22 herein), or functional variants thereof.

The crystalline anti-PCSK9 antibody can also be bococizumab (Pfizer RN316), described in U.S. Pat. No. 8,080,243 to Liang, et al. Bococizumab is effective at lowering LDL-cholesterol levels, and can be used in the prevention and/or treatment of cholesterol and lipoprotein metabolism disorders, including familial hypercholesterolemia, atherogenic dyslipidemia, atherosclerosis, and, more generally, CVD (cardiovascular disease). In one embodiment in the patent, the isolated antibody specifically binds to PCSK9 and comprises a heavy chain variable region (VH) complementary determining region one (CDR1) having the amino acid sequence shown in SEQ ID NO:8 (Ser Tyr Tyr Met His), 59 (Gly Tyr Thr Phe Thr Ser Tyr Tyr Met His), or 60 (Gly Tyr Thr Phe Thr Ser Tyr) (SEQ ID NOs: 23, 24, and 25 herein), a VH CDR2 having the amino acid sequence shown in SEQ ID NO:9 (Glu Ile Ser Pro Phe Gly Gly Arg Thr Asn Tyr Asn Glu Lys Phe Lys) or 61 (Ser Pro Phe Gly Gly Arg) (SEQ ID NOs: 26 and 27 herein), a VH CDR3 having the amino acid sequence shown in SEQ ID NO:10 (Glu Arg Pro Leu Tyr Ala Ser Asp Leu) (SEQ ID NO 28 herein), a light chain variable region (VL) CDR1 having the amino acid sequence shown in SEQ ID NO:11 (Arg Ala Ser Gln Gly Ile Ser Ser Ala Leu Ala) (SEQ ID NO: 29 herein), a VL CDR2 having the amino acid sequence shown in SEQ ID NO:12 (Ser Ala Ser Tyr Arg Tyr Thr) (SEQ ID NO: 30 herein), and a VL CDR3 having the amino acid sequence shown in SEQ ID NO:13 (Gln Gln Arg Tyr Ser Leu Trp Arg Thr) (SEQ ID NO: 31 herein). In another embodiment, the isolated antibody comprises a heavy chain variable region (VH) and a light chain variable region (VL), wherein the VH region comprises the amino acid sequence of SEQ ID NO: 54 (qvqlvqsgae vkkpgasvkv sckasgytft syymhwvrqa pgqglewmge ispfggrtny nekfksrvtm trdtststvy melsslrsed tavyycarer plyasdlwgq gttvtvss) (SEQ ID NO: 32 herein) and the VL region comprises the amino acid sequence of SEQ ID NO: 53 (diqmtqspss lsasvgdrvt itcrasqgis salawyqqkp gkapklliys asyrytgvps rfsgsgsgtd ftftisslqp ediatyycqq ryslwrtfgq gtkleik) (SEQ ID NO: 33 herein). In yet another embodiment, a humanized antibody comprises a light chain having the amino acid sequence of SEQ ID NO:14 (diqmtqspss lsasvgdrvt itcrasqgis salawyqqkp gkapklliys asyrytgvps rfsgsgsgtd ftftisslqp ediatyycqq ryslwrtfgq gtkleikrtv aapsvfifpp sdeqlksgta svvcllnnfy preakvqwkv dnalqsgnSq esvteqdskd styslsstlt lskadyekhk vyacevthqg lsspvtksfn rgec (SEQ ID NO: 34 herein) and a heavy chain having the amino acid sequence of SEQ ID NO:15 (qvqlvqsgae vkkpgasvkv sckasgytft syymhwvrqa pgqglewmge ispfggrtny nekfksrvtm trdtststvy melsslrsed tavyycarer plyasdlwgq gttvtvssas tkgpsvfpla pcsrstsest aalgclvkdy fpepvtvswn sgaltsgvht fpavlqssgl yslssvvtvp ssnfgtqtyt cnvdhkpsnt kvdktverkc cvecppcpap pvagpsvflf ppkpkdtlmi srtpevtcvv vdvshedpev qfnwyvdgve vhnaktkpre eqfnstfrvv svltvvhqdw lngkeykckv snkglpssie ktisktkgqp repqvytlpp sreemtknqv sltclvkgfy psdiavewes ngqpennykt tppmldsdgs fflyskltvd ksrwqqgnvf scsvmhealh nhytqkslsl spgk) (SEQ ID NO: 35 herein), with or without the C-terminal lysine of the amino acid sequence of SEQ ID NO: 15 (SEQ ID NO: 35 herein).

The crystalline antibody can also be any other suitable antibody in crystalline form, such as, but not limited to, 3F8, 8H9, abagovomab, abciximad, abrilumab, actoxumab, adalimumab, adecatumumab, aducanumab, afelimomab, afutuzumab, alacizumab pegol, ALD518, alemtuzumab, alirocumab, altumomab pentetate, amatuximab, anatumomab mafenatox, anifrolumab, anrukinzumab, apolizumab, arcitumomab, aselizumab, atinumab, atlizumab, atorolumumab, bapineuzumab, basiliximab, bavituximab, bectumomab, belimumab, benralizumab, bertilimumab, besilesomab, bevacizumab, beziotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cBR96-doxorubicin immunoconjugate, CC49, cedelizumab, certolizumab pegol, cetuximab, Ch.14.18, citatuzumab bogatox, cixutumumab, clazakizumab, clenoliximab, clivatuzumab tetraxetan, cnatumumab, concizumab, CR6261, crenezumab, dacetuzumab, daclizumab, dalotuzumab, daratumumab, demcizumab, denosumab, detumomab, dinutuximab, diridavumab, dorlimomab aritox, drozitumab, duligotumab, dupilumab, durvalumab, dusigitumab, ecromeximab, eculizumab, edobacomab, edrecolomab, efalizumab, efungumab, eldelumab, elotuzumab, elsilimomab, emibetuzumab, enavatuzumab, enfortumab vedotin, enlimomab pegol, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, ficlatuzumab, figitumumab, flanvotumab, fletikumab, fontolizumab, foralumab, forvirumab, fresolimumab, fluranumab, futuximab, galiximab, ganitumab, gantenerumab, gavilimomab, gemtuzumab ozogamicin, gevokizumab, girentuximab, glembatumumab vedotin, golimumab, gomiliximab, guselkumab, ibalizumab, ibritumomab tiuxetan, icrucumab, igovomab, IMAB362, imciromab, imgatuzumab, inclacumab, indatuximab ravtansine, infliximab, inolimomab, inotuzumab ozogamicin, intetumumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, lifastuzumab vedotin, ligelizumab, lintuzumab, lirilumab, lodelcizumab, lorvotuzumab, lorvotuzumab mertansine, lucatumumab, lulizumab pegol, lumiliximab, mapatumumab, margetuximab, maslimomab, matuzumab, mavrilimumab, mepolizumab, metelimumab, milatuzumab, minretumomam, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, obiltoxaximab, ocaratuzumab, ocrelizumab, odulimomab, ofatumumab, olaratumab, olokizumab, omalizumab, onartuzumab, ontuxizumab, oportuzumab monatox, oregovomab, orticumab, otelixizumab, otlertuzumab, oxelumab, ozanezumab, ozoralizumab, pagibaximab, palivizumab, panitumumab, pankomab, panobacumab, parsatuzumab, pascolizumab, pateclizumab, patritumab, pembrolizumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samilizumab, sarilumab, satumomab pendetide, secukinumab, seribantumab, setoxaximab, sevirumab, SGN-CD19A, SGN-CD33A, sibrotuzumab, sifalimumab, siltuximab, simtuzumab, siplizumab, sirukumab, sofitzumab vedotin, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tarextumab, tefibazumab, telimomab aritox, tenatumomab, teneliximab, teplizumab, teprotumumab, TGN1412, ticilimumab, tigatuzumab, tildrakizumab, TNX-650, tocilizumab, toralizumab, tositumomab, tovetumab, tralokinumab, trastuzumab, TRBS07, tregalizumab, tremelimumab, tucotuzumab celmoleukin, tuvirumab, ublituximab, urelumab, urtoxazumab, ustekinumab, vantictumab, vapaliximab, varlilumab, vatelizumab, vedolizumab, veltuzumab, vepalimomab, vesencumab, volociximab, vorsetuzumab mafodotin, votumumab, zalutumumab, zanolimumab, zatuximab, ziralimumab, or zolimimab aritox.

The crystalline antibody can be formulated with various excipients and carriers that aid in the preservation of the crystal until a desired release point in the body (preferably in the gut). In other words, the composition can be formulated to protect the crystalline antibody (as well as the half-life-extending compound) from degradation until it reaches the gut of the individual. A polymeric carrier can be used and include one or more polymer of poly(acrylic acid), poly(cyanoacrylates), poly(amino acids), poly(anhydrides), poly(depsipeptide), poly(esters), poly(lactic acid), poly(lactic-co-glycolic acid) or PLGA, poly(β-hydroxybutryate), poly(caprolactone), poly(dioxanone); poly (ethylene glycol), poly(hydroxypropyl)methacrylamide, poly(organo) phosphazene, poly(ortho esters), poly(vinyl alcohol), poly(vinylpyrrolidone), maleic anhydride alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. An oil (or oily liquid) carrier can be one or more of oleaginous almond oil, corn oil, cottonseed oil, ethyl oleate, isopropyl myristate, isopropyl palmitate, mineral oil, light mineral oil, octyldodecanol, olive oil, peanut oil, persic oil, sesame oil, soybean oil, squalane, liquid triglycerides, liquid waxes, and higher alcohols. A lipid carrier can be one or more lipid of fatty acids and salts of fatty acids, fatty alcohols, fatty amines, mono-, di-, and triglycerides of fatty acids, phospholipids, glycolipids, sterols and waxes and related similar substances.

The most preferred combination of the composition of the present invention is lactulose and crystalline anti-PCSK9 MAb, and more specifically, lactulose and crystalline bococizumab.

It should be understood that the mechanism of action of the half-life-extending compound can be through the conversion of NH3 to NH4+ in combination with or in the alternative to the generation of hydrogen. The same compounds can accomplish both mechanisms, or different compounds can be used for the half-life-extending compound to accomplish each individual mechanism when and where desired.

The half-life-extending compound can be delivered at the same time as the crystalline antibody, or at different times. The half-life-extending compound can be contained within its own dosage form, within the dosage form together with the crystalline antibody (i.e. a capsule containing the half-life-extending compound and the crystalline antibody), or within the dosage form itself (i.e. a capsule coating that includes the half-life-extending compound, with the crystalline antibody within the capsule). The composition of the present invention can be tailored to provide different release profiles as needed or desired for a particular patient, such as, but not limited to, sustained release, prolonged release, or immediate release. The half-life-extending compound and the crystalline antibody can each have the same release profiles or different release profiles.

The crystalline antibody and half-life-extending compound act in a synergistic manner. Therefore, the antibody is preferably present in an amount that is lower than the normal effective dose. The half-life-extending compound can also be present in an amount that is lower than the normal effective dose. In other words, by combining the crystalline antibody with the half-life-extending compound, the effective amount needed can be reduced, which in turn reduces unwanted side effects. Therefore, the crystalline antibody and the half-life-extending compound can be present in synergistically effective amounts. This combination also allows for the use of antibodies that have previously had too short a half-life to be effective in the body. Hydrogen generated by the hydrogen-generating compound lactulose has showed an additive effect on pregabalin-mediated suppression of pain associated factors (Substance P and nerve growth factor (NGF)) in mice. Because the half-life of the crystalline antibody is increased, its activity is increased, and a smaller dose can be used to achieve the same activity as in previous administration. Also, because the crystalline antibody can be delivered protected to the gut in an oral form, it can uniquely interact with the half-life-extending compound in the gut to produce the synergistic effects. The half-life extending compound can further provide effects of reducing/eliminating inflammation and positively altering lipid metabolism.

The composition of the present invention can be delivered alone or in combination with additional agents. For example, the composition can be combined with statins that lower cholesterol levels in blood and prevent heart attacks and strokes, such as, but not limited to, atorvastatin, cerivastatin, fluvastain, lovastatin, mevastatin, pitavastatin, pravastatin, rosuvastatin, or simvastatin. The agent can be non-steroidal anti-inflammatory drugs (NSAIDS) such as, but not limited to, acetaminophen, salicylates (aspirin, diflunisal, salsalate), acetic acid derivatives (indomethacin, ketorolac, sulindac etodolac, diclofenac, nabumetone), propionic acid derivatives (ibuprofen, naproxen, flurbiprofen, ketoprofen, oxaprozin, fenoprofen, loxoprofen), fenamic acid derivatives (meclofenamic acid, mefenamic acid, flufenamic acid, tolfenamic acid), oxicam (enolic acid) derivatives (piroxicam, meloxicam, tenoxicam, droxicam, lornoxicam, isoxicam), arylalkanoic acid derivatives (tolmetin); or selective COX-2 inhibitors (celecoxib, rofecoxib, valdecoxib, parecoxib, lumiracoxib, etoricoxib, firocoxib). The agent can also be generally from the classes antihistamines, anti-infective agents, antineoplastic agents, autonomic drugs, blood derivatives, blood formation agents, coagulation agents, thrombosis agents, cardiovascular drugs, cellular therapy, central nervous system agents, contraceptives, dental agents, diagnostic agents, disinfectants, electrolytic, caloric, and water balance, enzymes, respiratory tract agents, eye, ear, nose, and throat preparations, gold compounds, heavy metal antagonists, hormones and synthetic substitutes, oxytocics, radioactive agents, serums, toxoids, and vaccines, skin and mucous membrane agents, smooth muscle relaxants, and vitamins.

The composition of the present invention can be used for treating many different diseases and conditions in which antibodies are generally administered. These diseases and conditions can be, but are not limited to, asthma, autoimmune diseases, rheumatoid arthritis, transplant rejection, allergies, cancers (including neuroblastoma, sarcoma, metastatic brain cancer, ovarian cancer, prostate cancer, breast cancer, lymphoma, colorectal cancer, non-small cell lung carcinoma, hematological cancers, gastrointestinal cancers, squamous cell carcinoma, pancreatic cancer, and melanoma), Crohn's disease, Hodgkin disease, platelet diseases, inflammatory bowel disease, ulcerative colitis, infections, psoriasis, arthritis, ankylosing spondylitis, hemolytic disease, Alzheimer's disease, sepsis, Multiple sclerosis, hypercholesterolemia, systemic lupus erythematosis, inflammation, thromboembolism, bleeding, infectious diseases, osteoporosis, hepatitis B, dyslipidemia, disease diagnosis, leukemia, pain, diabetes, or HIV. Any of these diseases can be treated according to the methods detailed below.

The present invention provides for a method of extending the half-life of a crystalline antibody, by administering a synergistically effective amount of the composition including the half-life-extending compound of the non-absorbable sugar, compound that converts NH3 to NH4+, prebiotic, or hydrogen-generating compound and the crystalline antibody to an individual, and extending the half-life of the crystalline antibody. The half-life-extending compound can be any of those described above, and preferably lactulose. The crystalline antibody can be any of those described above, and preferably a crystalline anti-PCSK9 mAb, and especially crystalline bococizumab. Administration can be oral, by injection, topical, or any other administration profile described herein. Preferably, administration of both the half-life-extending compound and the crystalline antibody is oral. This method is unique in providing oral administration of antibodies in any of the forms and delivery profiles described above, whereas previously antibodies needed to be delivered by i.v. infusion or s.c. injection. Alternatively, the half-life-extending compound can be administered orally and the crystalline antibody can be administered by injection. The half-life-extending compound and the crystalline antibody preferably act synergistically to extend the half-life of the crystalline antibody. Because the half-life is increased, the activity of the crystalline antibody can be increased. The half-life is extended by the interaction of the half-life extending compound and the crystalline antibody by promoting FcRn-receptor mediated pH-dependent IgG recycling process at gut epithelium. More specifically, in the case of the anti-PCSK9 mAb, the half-life is extended by dissociating the anti-PCSK9 mAb/PCSK9 immune complex during trans-epithelial trafficking of the immune complex of PCSK9-bound anti-PCSK9 mAb. The half-life-extending compound can also reduce and/or eliminate inflammation as described above, as well as alter lipid metabolism and provide these effects synergistically in combination with the crystalline antibody. The crystalline antibody itself can also alter lipid metabolism as described above. It should also be understood that in a crystalline form, higher concentrations of the antibody can be administered than in non-crystalline form (i.e. 200 mg/ml or more versus 100 mg/ml), especially with injection. However, due to the synergy with the half-life-extending compound, lower doses can be used.

More generally, the present invention provides for a method of promoting antibody recycling by affecting a receptor for an antibody to recycle the antibody. The receptor is affected by creating an acidic environment in a body (preferably in the gut or GI tract). Preferably, the acidic environment is less than pH 7.4, and more preferably, the pH is 6.0 or less. The acidic environment is created by administering the half-life-extending compound of the non-absorbable sugar, compound that converts NH3 to NH4+, prebiotic, or hydrogen-generating compound. The half-life-extending compound can be any of those described above, and preferably lactulose. The half-life-extending compound is administered along with a crystalline antibody. The crystalline antibody can be any of those described above, and preferably a crystalline anti-PCSK9 mAb, and especially crystalline bococizumab. Administration can be oral, by injection, topical, or any other administration profile described herein. Most preferably, the half-life-extending compound and the crystalline antibody are administered orally. With oral administration preferably, the crystalline antibody is protected from degradation until reaching the individual's gut where it can interact with the half-life-extending compound under the influence of the gut microbiome, as described above. The crystalline antibody is more effectively resorbed at this point and can reach an effective dose and maintain that effective dose. Alternatively, the half-life-extending compound can be administered orally and the crystalline antibody can be administered by injection. The half-life-extending compound and the crystalline antibody preferably act synergistically to affect the receptor to recycle the antibody by unconjugating the antibody, allowing recirculation, and therefore extending the half-life of the crystalline antibody.

The compounds of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Synopsis

Oral administration of lactulose extends the serum half-life of anti-PCSK9 mAb via promoting FcRn-receptor mediated pH-dependent IgG recycling process at gut epithelium. Such an FcRn-mediated anti-PCSK9 mAb recycling appeared to be attained by the biological mechanism that dissociates anti-PCSK9 mAb/PCSK9 immune complex during trans-epithelial trafficking of the immune complex of PCSK9-bound anti-PCSK9 mAb. Most importantly, because the elimination of gut bacteria with systemic administration of antibiotics abolished the lactulose's effects on extending the serum half-life of anti-PCSK9 mAb, the prebiotic effects of lactulose that help acid production by gut commensal bacteria seemed to play a key role in this pH-dependent IgG recycling. These data support that oral administration of lactulose can promote the serum half-life of not only anti-PCSK9 mAb but also other mAb drugs.

Materials and Methods

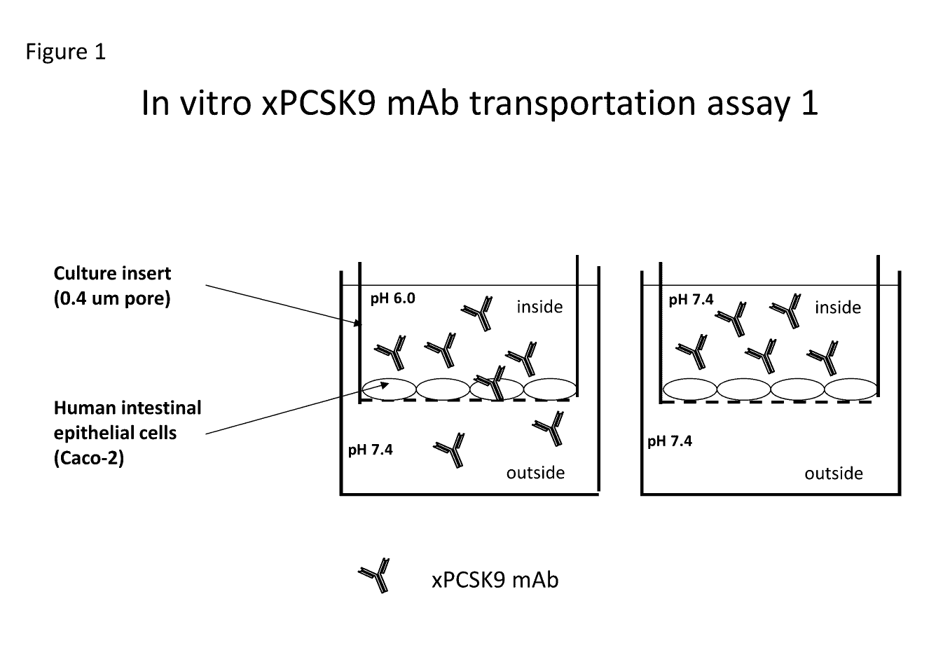

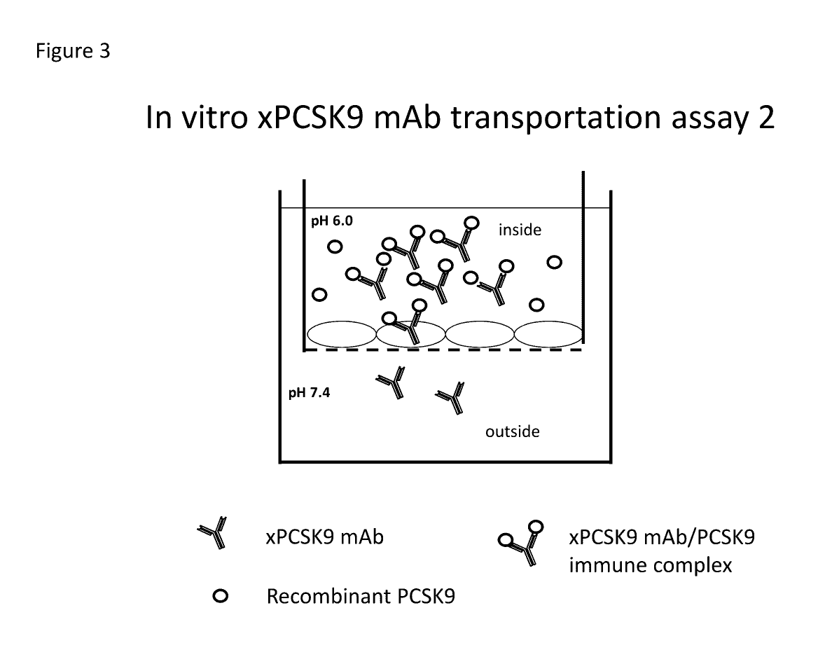

Trans-Epithelial xPCSK9-mAb Transport Assay (FIGS. 1 and 3):

Immortalized mouse epithelial cell line (GE1) was employed for examination of in vitro trans-epithelial transportation of xPCSK9-mAb (mouse IgG3, Pfizer). GE1 cells were cultured inside of tissue culture insert (Corning, 0.4 μM pore size) that was place in the well of 24 well-plate using a-MEM containing 10% FBS, Non-Essential Amino Acids Solution (Life technology), antibiotics and L-glutamine (a-MEM complete medium), After GE1 cells reached the confluence in the culture insert, the trans-epithelial xPCSK9-mAb transport assay was carried out.

The xPCSK9-mAb was labeled with biotin using EZ-Link Sulfo-NHS-LC-Biotin conjugation kit (Thermo Fisher Scientific). Biotin-xPCSK9-mAb suspended in aMEM complete medium of which pH was adjusted at T4 or 6.0 was applied into the culture insert with confluent GE1 cells. Subsequently, those culture inserts containing biotin-xPCSK9-mAb and confluent GE1 cells were placed into the well of 24-well plate that contain aMEM complete medium of which pH was adjusted at either 7.4 or 6.0. As a result, there were a total four different combinations of pH prepared in the inside of culture insert and outside compartment of culture insert, respectively, as follows (inside vs. outside): 1) 7.4 vs. 7.4, 2) 6.0 vs. 7.0, 3) 6.0 vs. 6.0 and 4) 7.4 vs. 6.0.

The amount of biotin-xPCSK9-mAb that transposed through the Caco-2 monolayer on the culture insert and reached to outside compartment was monitored using direct ELISA system. More specifically, recombinant mouse PCSK9 (Biolegend) or goat anti-mouse IgG (fab fraction, Jackson immunoresearch) was coated on the wells of ELISA plate (2 ug/ml, respectively, in pH9.4 bicarbonate buffer). Subsequently, the sample containing biotin-xPCSK9-mAb that was collected from outside compartment of culture insert was applied to the ELISA plate. In some wells, serially diluted biotin-xPCSK9 mAb was applied as standard for the titration. The bound biotin-xPCSK9 mAb to the well of ELISA plate was reacted with Avidin-conjugated horse radish peroxidase (HRPO). The HRPO-mediated substrate color development was carried out by the addition of o-Phenylenediamine (OPD) dissolved in 0.1 M sodium phosphate/citric acid buffer (pH 5.0) containing 6 mM H2O2.

In some experiments, anti-PCSK9-mAb was reacted with excess amount of PCSK9 so that all anti-PCSK9-mAb form immune complex with PCSK9. The immune complex between anti-PCSK9-mAb and recombinant PCSK9 was applied to culture insert with confluent GE1 cells in pH 7.4 medium, which was then placed into the well of 24-well-pate that contain pH 6.0 medium. In this manner, it was examined if transposition of anti-PCSK9-mAb/PCSK9 immune complex through GE1 monolayer can dissociate the mAb from the complex.

Evaluation of Effects of Lactulose on Serum Half-Life of xPCSK9-mAb Using an In Vivo Mouse Model:

At Day 0, anti-PCSK9-mAb was systemically administrated (30 mg/kg in PBS, i.v.) to the FcRn gene knockout mice or their wild type C57BL/6 mice (8 w old male). Lactulose (30%, water solution) was orally administered using a Popper's feeding needle once in a day throughout the experiment. In some experiment, groups of mice received antibiotics cocktail (Ampicillin, 2 mg/ml; Vancomycin, 1 mg/ml, Metronidazole 2 mg/ml and Neomycin, 2 mg/ml) ad libitum for 7 days. Blood was collected from mice at Day 1 and Day 7 (100 ul/mouse) by retro-orbital bleeding. The serum was separated from the collected blood and saved at −70 C until the measurement of anti-PCSK9-mAb as well as low-density lipoprotein (LDL) cholesterol in the sampled serum. At Day 7, right after the collection of blood, mice were sacrificed, and liver, small intestine (distal portion, 2 cm above cecum) and large intestine (proximal colon, 1 cm below cecum) were collected. The collected liver was homogenized in PBS containing Tween 20 (0.5%) and proteinase inhibitor cocktail (Sigma). For the small and large intestines, the contents in the lumen as well as a layer of epithelium was removed and homogenized in the same solution used for liver. The amount of LDL/VLDL in the serum was monitored using a HDL and LDL/VLDL Quantitation Kit (Sigma-Aldrich). The amount of anti-PCSK9-mAb in serum, liver, small and large intestines were measured using the ELISA as described above #1. The luminal pH level of the large intestine was measure by dissolving the luminal contents in distilled water (0.2 g/1 ml water).

In some experiments, biotin-conjugated xPCSK9-mAb was adoptively transferred into mice. The amounts of free xPCSK9-mAb and antigen (PCSK9) bound xPCSK9-mAb present in intestines were monitored. For the detection of total biotin-conjugated xPCSK9-mAb (free xPCSK-mAb and xPCSK9-MAb/PCSK9 immune complex), goat anti-mouse IgG polyclonal antibody (Goat IgG Fab, Jackson Immunoreserach) was coated on the 96 ELISA plate to capture the IgG present in the contents of intestine, followed by HRPO-conjugated avidin. This ELISA system detected both free and PCSK9-bound xPCSK9 mAb (=total xPCSK9 mAb) in intestines. For the detection of free biotin-conjugated-xPCSK9-mAb, mouse recombinant PCSK9 was coated on the 96 ELISA plate to capture the free xPCSK9-mAb. In both ELISA systems, HRPO-conjugated-avidin was used for quantification of biotin-conjugated-xPCSK9-mAb using OPD/H2O2 substrate.

Results

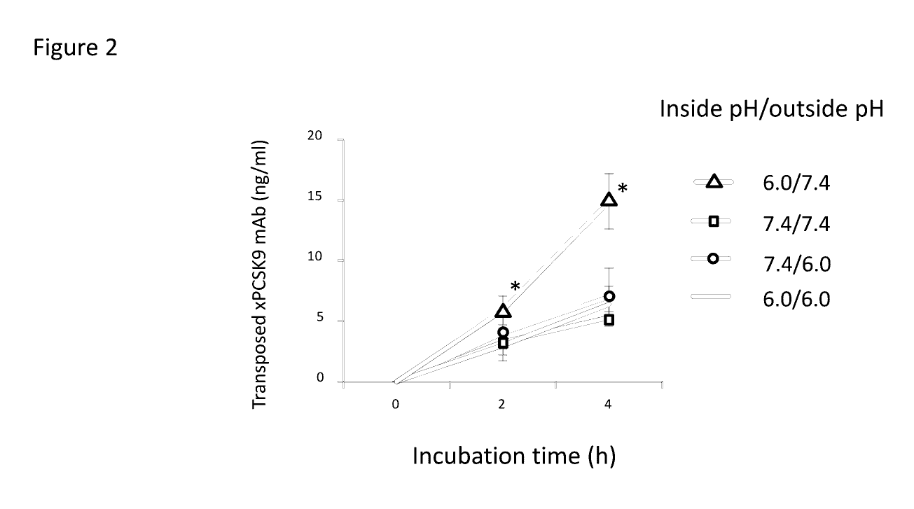

xPCSK9-mAb is Transposed Through Cultured Epithelial Cells in a pH Gradient Dependent Manner (FIG. 2):

xPCSK9-mAb (10 ug/ml) was applied into the culture insert that had confluent monolayer of human intestinal epithelial cells (Caco-2) at different pH, either 6.0 or 7.4. Then, the culture insert was further placed into the well of 24-well plate that contain medium adjust at pH 6.0 or 7.4. After incubation for 2 and 4 hours, the medium in the outside of the culture insert was harvested and subjected to ELISA that monitored the amount of transposed xPCSK9 mAb.

The xPCSK9-mAb applied to culture insert with confluent GE1 cells containing medium at pH 6.0 showed significantly elevated transposition to the outside compartment that had the medium at pH 7.4, but not to pH 6.0. However, the xPCSK9-mAb in the culture insert containing medium at pH 7.4 did not show such an elevated transposition to the outside compartment containing the medium at pH 7.4 or 6.0. This result indicated that xPCSK9-mAb can be transposed from acidic side (pH 6.0) to neutral side (pH 7.4) through epithelial cell layer.

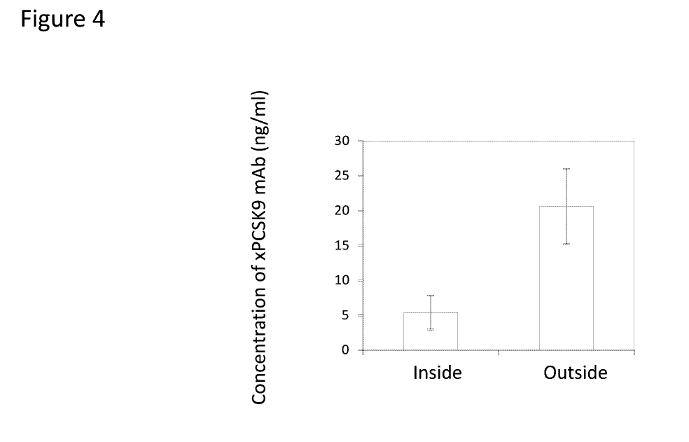

Possible Dissociation of xPCSK9-mAb/PCSK9 Immune Complex as a Consequence of Trans-Epithelial Transport (FIG. 4):

The xPCSK9-mAb (20 ug/ml, MW 150 kD) was reacted with the recombinant mouse PCSK9 (20 ug/ml, MW 74 kD) in culture medium (pH 6.0) for 1 hour so that an immune complex between xPCSK9-mAb and PCSK9 can be formed. The immune complex of xPCSK9-mAb/PCSK9 in medium was applied to the culture insert that had confluent GE1 cells (FIG. 3) which was then placed into the well of 24-well plate that contains medium adjusted at 7.4. After incubation for 4 hours, the amount of free xPCSK9-mAb was monitored using a direct ELISA in which recombinant mouse PCSK9 is coated as antigen. The amount of free xPCSK9-mAb was significantly higher in outside compartment than that of inside of culture insert, showing that xPCSK9-mAb/PCSK9 immune complex can be dissociated during the trans-epithelial transportation.

Promotion of Serum Half-Life of xPCSK9 mAb in Mice by Oral Administration of Lactulose:

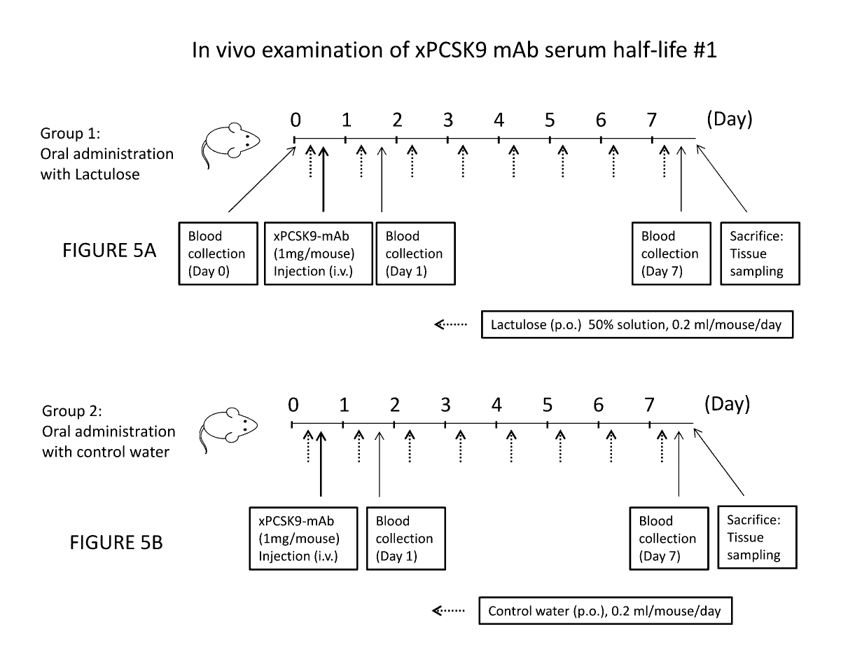

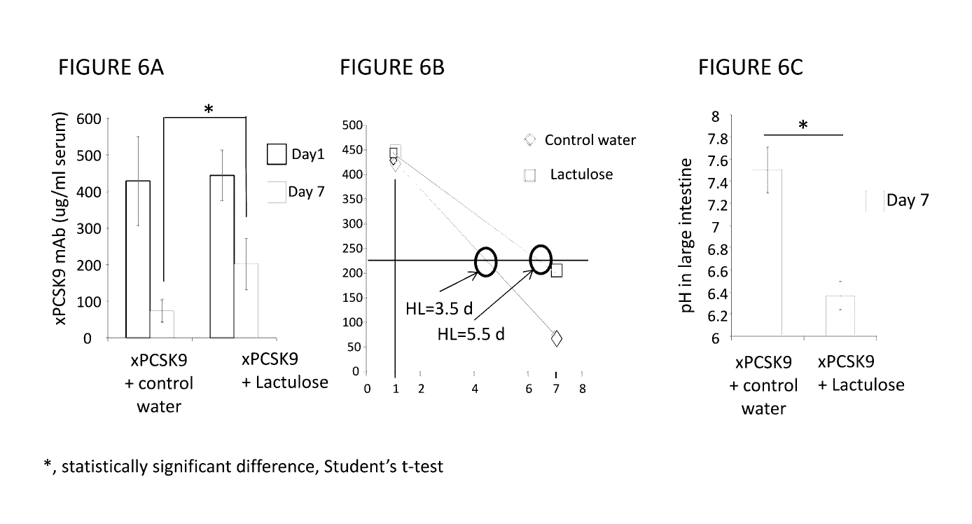

The xPCSK9-mAb (xPCSK9-mAb 1 mg/mouse) was injected into C57BL6 mice at Day 0. From Day-0 to Day 7, Lactulose (50% water solution, 0.2 ml/mouse) or control water (0.2 ml/mouse) was orally administered once/day (see FIGS. 5A and 5B). At Day 1 and Day 7, blood serum was collected and the amount of xPCSK9-mAb was monitored using direct ELISA. For the ELISA, blood serum diluted in PBS with 0.1% Tween 20 was reacted with recombinant PCSK9 that was directly coated on 96-well ELISA plate. Subsequently, HRPO-conjugated anti-mouse IgG was applied into the reaction well, followed by colorimetric reaction using substrate solution containing OPD/H2O2. The amounts of xPCSK9 mAb detected in serum at day 1 and Day 7 are shown (FIG. 6A). The daily oral administration of lactulose significantly elevated the amount of xPCSK9 remaining in the blood serum at Day 7 compared to the control group that received water. Calculated serum Half-Life (HL) for control group was 3.5 day, whereas that for group received Lactulose was 5.5 day (FIG. 6B). Content of large intestine sampled from sacrificed mice was suspended in water (0.1 g/1 ml) and the pH was monitored (FIG. 6C).

Lactulose Did not Affect the Serum Concentration of LDL in the Mice that Received xPCSK9 mAb:

The xPCSK9-mAb (xPCSK9-mAb 1 mg/mouse) was injected into C57BL6 mice at Day 0. From Day-0 to Day 7, Lactulose (50% water solution, 0.2 ml/mouse) or control water (0.2 ml/mouse) was orally administered once/day. One group of mice received only daily lactulose without injection of xPCSK9. At Day 0, prior to any administrations, and Day 7, blood serum was collected and the concentration of LDL was monitored using LDL detection kit (Sigma).

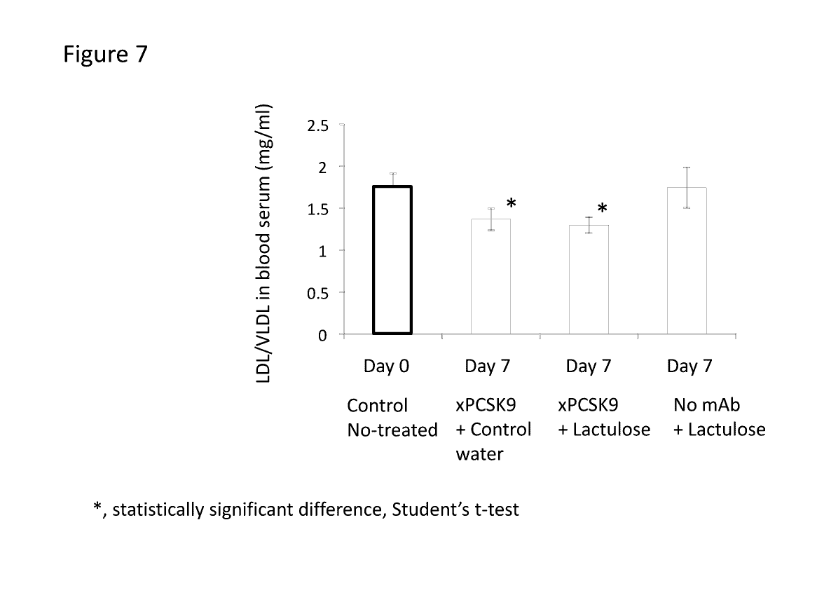

In order to determine the functionality of xPCSK9 injected into mice, the level of LDL in blood serum was measure at Day 7 (FIG. 7). The group that received xPCSK9 and control water as well as that received xPCSK9 and lactulose showed the suppression of LDL level in the blood serum compared to the base line control serum which was sampled prior to the administration with reagents (Day 0). There was no statistical difference between the LDL levels on those two groups that received xPCSK9 mAb. The mice that received oral lactulose alone without xPCSK9 mAb did not show any changes from the base line level. It is noteworthy that in another study using a mouse model of high fat diet induced hyperlipidemia, it was found that lactulose can suppress oxidized LDL (oxLDL), but not LDL.

Engagement of FcRn in the Retention of xPCSK9-mAb in Mouse Blood Serum:

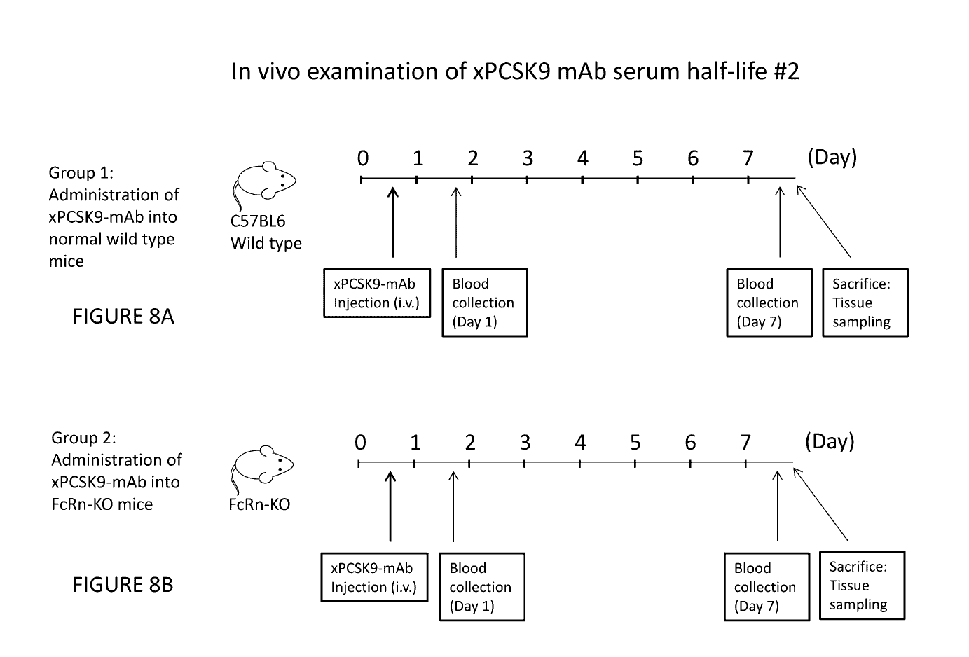

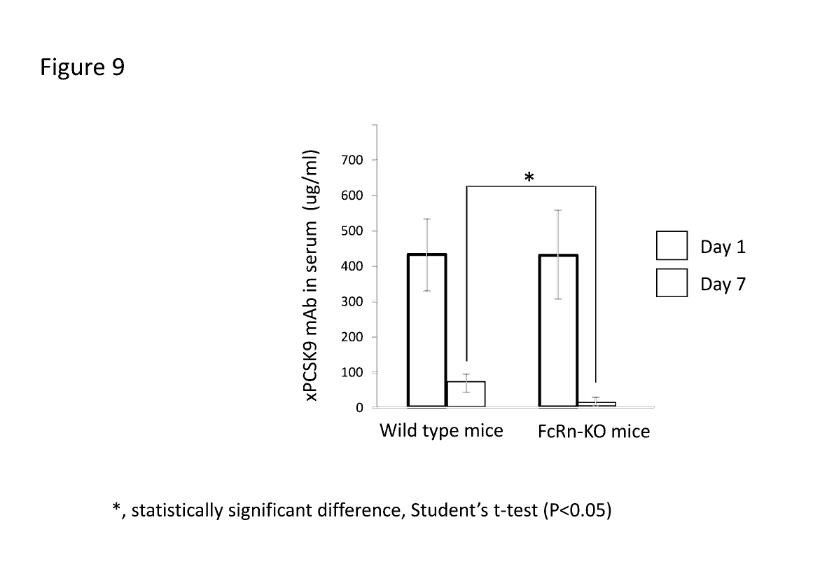

The xPCSK9-mAb (xPCSK9-mAb 1 mg/mouse) was injected into FcRn-KO mice or their wild type control mice (C57BL6) at Day 0 (see FIGS. 8A and 8B). At Day 1 and Day 7, blood serum was collected and the amount of xPCSK9-mAb was monitored using a direct ELISA (FIG. 9). For the ELISA, blood serum diluted in PBS with 0.1% Tween 20 was reacted with recombinant PCSK9 that was directly coated on 96-well ELISA plate. Subsequently, HRPO-conjugated anti-mouse IgG was applied into the reaction well, followed by colorimetric reaction using substrate solution containing OPD/H2O2. The amount of xPCSK9 mAb detected in FcRn-KO mice were significantly lower than that found in wild type mice, showing that FcRn is engaged in the xPCSK9 recycling process in the mice.

Requirement of Gut Bacteria for the Lactulose to Increase the Serum Half-Life of xPCSK9 mAb:

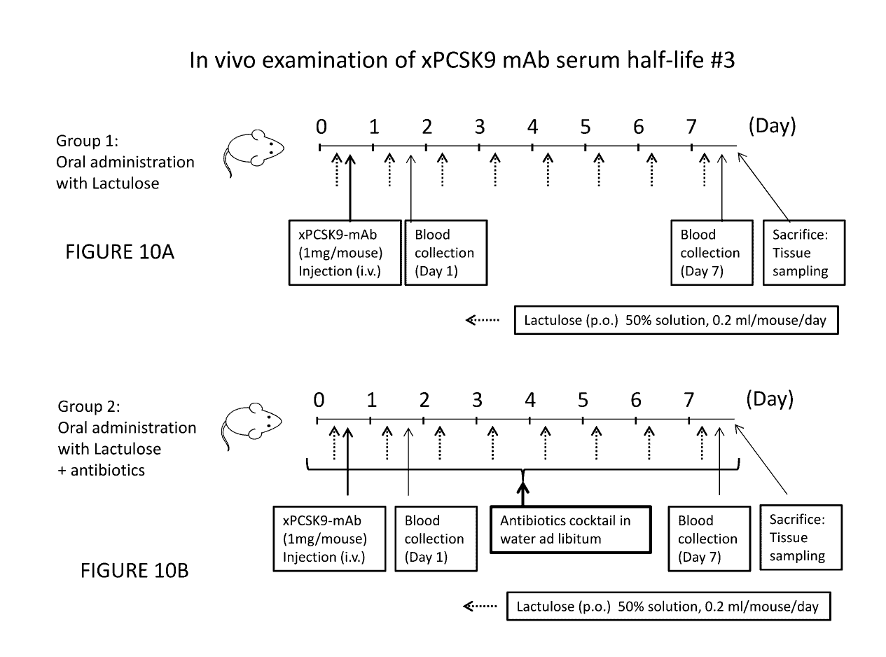

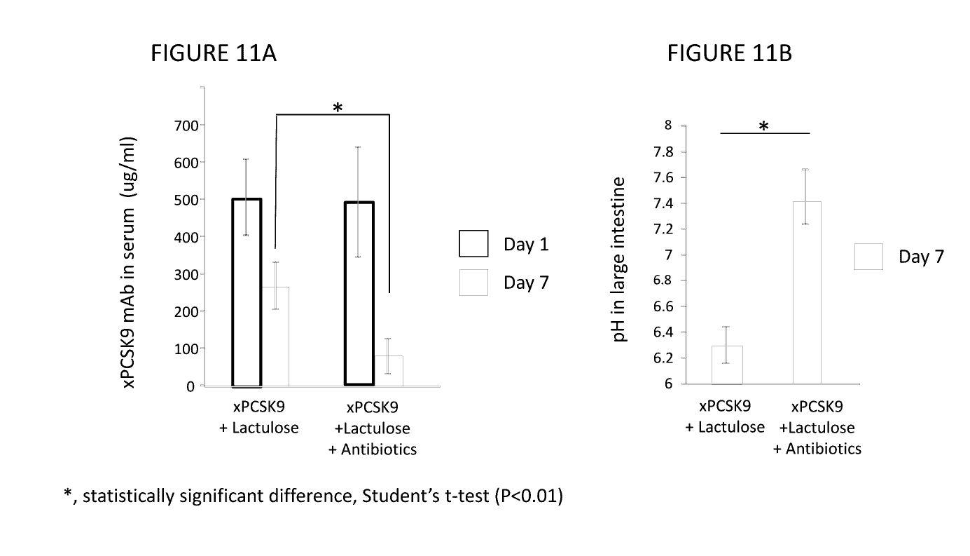

In order to examine the role of gut bacteria for the lactulose-mediated extension of xPCSK9 mAb's serum half-life in mice, a group of mice was administered with antibiotics cocktail via drinking water (FIG. 10B), while the other group was maintained with regular water (FIG. 10A). The xPCSK9-mAb (xPCSK9-mAb 1 mg/mouse, i.v.) was injected into C57BL6 mice at Day 0. From Day-0 to Day 7, Lactulose (50% water solution, 0.2 ml/mouse) was orally administered once/day. One group of mice further received antibiotics cocktail in drinking water ad libitum. At Day 1 and Day 7, blood serum was collected and the amount of xPCSK9-mAb was monitored using a direct xPSCK9-mAb detection ELISA. In FIG. 11A, the amounts of xPCSK9 mAb detected in serum at day 1 and Day 7 are shown. In FIG. 11B, content of large intestine sampled from sacrificed mice was suspended in water (0.1 g/1 ml) and the pH was monitored.

Very interestingly, the effect of lactulose on extending the serum half-life was abolished by the treatment with the antibiotics cocktail. Furthermore, lactulose's effect to lower the pH of large intestine was also abrogated and became neutral range (pH 7.4) by antibiotics treatment. These results indicated that 1) pH lowering effects by lactulose is derived from its influence on gut bacteria, and 2) the acidification of gut lumen caused by lactulose is responsible for the FcRn mediated xPCSK9 recycling from large intestine.

Possible Dissociation of PCSK9 Bound xPCSK9-mAb Immune Complex in Large Intestine:

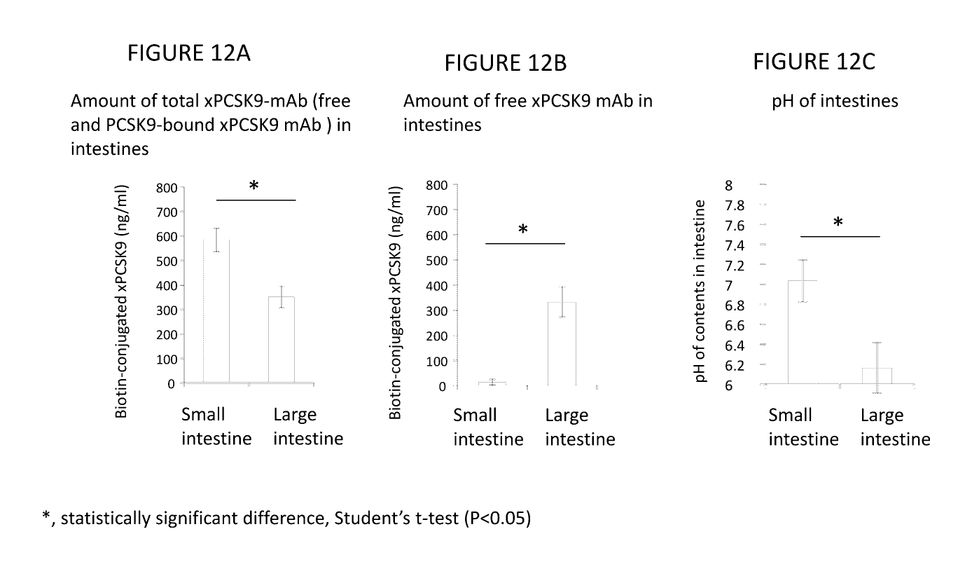

In order to investigate if and where xPCSK9 transposition occurs in gut, biotin-conjugated xPCSK9-mAb (xPCSK9-mAb 1 mg/mouse, i.v.) was injected into C57BL6 mice at Day 0. From Day-0 to Day 2, lactulose (50% water solution, 0.2 ml/mouse) was orally administered once/day. Two days after the injection of biotin-conjugated xPCSK9-mAb, the mice were sacrificed. Subsequently, small and large intestines were collected and the contents including epithelium were suspended in PBS+ tween 20 (for ELISA) or water (for pH). Goat anti-mouse IgG polyclonal antibody (Goat IgG Fab, Jackson Immunoreserach) was coated on the 96 ELISA plate to capture the IgG present in the contents of intestine, followed by HRPO-conjugated avidin. This ELISA system detects both free and PCSK9-bound xPCSK9 mAb (=total xPCSK9 mAb) in intestines. Mouse recombinant PCSK9 was coated on the 96 ELISA plate to capture the free xPCSK9-mAb. The pH of contents in large and small intestines were monitored. The amount of biotin-conjugated total xPCSK9-mAb that involve both free-xPCSK9 and PCSK9-bound xPCSK9-was higher in small intestine than large intestine (FIG. 12A). However, the amount of free xPCSK9-mAb was higher in large intestine than small intestine (FIG. 12B). Furthermore, pH of the large intestine was lower (=acidic) than the small intestine (FIG. 12C). These results show that the PCSK9-bound xPCSK9-mAb immune complex is present in the small intestine, and such an immune complex is dissociated in the large intestine.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Devanaboyina S C, Lynch S M, Ober R J, Ram S, Kim D, Puig-Canto A et al. (2013). The effect of pH dependence of antibody-antigen interactions on subcellular trafficking dynamics. mAbs 5(6):851-859.
Florent C, Flourie B, Leblond A, Rautureau M, Bernier J J, Rambaud J C (1985). Influence of chronic lactulose ingestion on the colonic metabolism of lactulose in man (an in vivo study). The Journal of clinical investigation 75(2):608-613.
Hornby P J, Cooper P R, Kliwinski C, Ragwan E, Mabus J R, Harman B et al. (2014). Human and non-human primate intestinal FcRn expression and immunoglobulin G transcytosis. Pharmaceutical research 31(4):908-922.
Poirier S, Mayer G (2013). The biology of PCSK9 from the endoplasmic reticulum to lysosomes: new and emerging therapeutics to control low-density lipoprotein cholesterol. Drug design, development and therapy 7 (1135-1148.
Sahota S S, Bramley P M, Menzies I S (1982). The fermentation of lactulose by colonic bacteria. Journal of general microbiology 128(2):319-325.
Salminen S, Salminen E (1997). Lactulose, lactic acid bacteria, intestinal microecology and mucosal protection. Scandinavian journal of gastroenterology Supplement 222 (45-48.
Yoshida M, Claypool S M, Wagner J S, Mizoguchi E, Mizoguchi A, Roopenian D C et al. (2004). Human neonatal Fc receptor mediates transport of IgG into luminal secretions for delivery of antigens to mucosal dendritic cells. Immunity 20(6):769-783.

What is claimed is:

1. A composition for extending the half-life of a crystalline antibody, comprising synergistically effective amounts of a half-life-extending compound and a crystalline antibody.

2. The composition of claim 1, wherein said half-life-extending compound is chosen from the group consisting of a non-absorbable sugar, a compound that converts NH3 to NH4+, a prebiotic, a hydrogen-generating compound, and combinations thereof.

3. The composition of claim 2, wherein said half-life-extending compound is lactulose or homologues thereof and is in an amount lower than indicated for treating constipation, said amount being less than 10 g/day.

4. The composition of claim 2, wherein said non-absorbable sugar is chosen from the group consisting of lactulose, glucose, galactose, fructose, mannitol, inulin, sucralose, aspartame, dextrose, maltodextrin, homologues, and combinations thereof.

5. The composition of claim 2, wherein said prebiotic is chosen from the group consisting of lactulose, disaccharides, inulin, fructooligosaccharides (FOS), xylooligosaccharides (XOS), polydextrose, oligofructose-enriched inulin, resistant starch, galactooligosaccharides (GOS), transgalactooligosaccharide, mannooligosaccharides, tagatose, and monosaccharides.

6. The composition of claim 2, wherein said hydrogen-generating compound is chosen from the group consisting of H₂ molecules, a prodrug releasing H₂, a compound that causes the release of H₂ within the body, an H₂ infused liquid, and combinations thereof.

7. The composition of claim 2, wherein said compound that converts NH3 to NH4+ is lactulose or homologues thereof.

8. The composition of claim 1, wherein said crystalline antibody is crystalline anti-PCSK9 MAb.

9. The composition of claim 8, wherein said crystalline anti-PCSK9 MAb is crystalline bococizumab.

10. The composition of claim 1, wherein said crystalline antibody is chosen from the group consisting of 3F8, 8H9, abagovomab, abciximad, abrilumab, actoxumab, adalimumab, adecatumumab, aducanumab, afelimomab, afutuzumab, alacizumab pegol, ALD518, alemtuzumab, alirocumab, altumomab pentetate, amatuximab, anatumomab mafenatox, anifrolumab, anrukinzumab, apolizumab, arcitumomab, aselizumab, atinumab, atlizumab, atorolumumab, bapineuzumab, basiliximab, bavituximab, bectumomab, belimumab, benralizumab, bertilimumab, besilesomab, bevacizumab, beziotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cBR96-doxorubicin immunoconjugate, CC49, cedelizumab, certolizumab pegol, cetuximab, Ch.14.18, citatuzumab bogatox, cixutumumab, clazakizumab, clenoliximab, clivatuzumab tetraxetan, cnatumumab, concizumab, CR6261, crenezumab, dacetuzumab, daclizumab, dalotuzumab, daratumumab, demcizumab, denosumab, detumomab, dinutuximab, diridavumab, dorlimomab aritox, drozitumab, duligotumab, dupilumab, durvalumab, dusigitumab, ecromeximab, eculizumab, edobacomab, edrecolomab, efalizumab, efungumab, eldelumab, elotuzumab, elsilimomab, emibetuzumab, enavatuzumab, enfortumab vedotin, enlimomab pegol, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, ficlatuzumab, figitumumab, flanvotumab, fletikumab, fontolizumab, foralumab, forvirumab, fresolimumab, fluranumab, futuximab, galiximab, ganitumab, gantenerumab, gavilimomab, gemtuzumab ozogamicin, gevokizumab, girentuximab, glembatumumab vedotin, golimumab, gomiliximab, guselkumab, ibalizumab, ibritumomab tiuxetan, icrucumab, igovomab, IMAB362, imciromab, imgatuzumab, inclacumab, indatuximab ravtansine, infliximab, inolimomab, inotuzumab ozogamicin, intetumumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, lifastuzumab vedotin, ligelizumab, lintuzumab, lirilumab, lodelcizumab, lorvotuzumab, lorvotuzumab mertansine, lucatumumab, lulizumab pegol, lumiliximab, mapatumumab, margetuximab, maslimomab, matuzumab, mavrilimumab, mepolizumab, metelimumab, milatuzumab, minretumomam, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, obiltoxaximab, ocaratuzumab, ocrelizumab, odulimomab, ofatumumab, olaratumab, olokizumab, omalizumab, onartuzumab, ontuxizumab, oportuzumab monatox, oregovomab, orticumab, otelixizumab, otlertuzumab, oxelumab, ozanezumab, ozoralizumab, pagibaximab, palivizumab, panitumumab, pankomab, panobacumab, parsatuzumab, pascolizumab, pateclizumab, patritumab, pembrolizumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samilizumab, sarilumab, satumomab pendetide, secukinumab, seribantumab, setoxaximab, sevirumab, SGN-CD19A, SGN-CD33A, sibrotuzumab, sifalimumab, siltuximab, simtuzumab, siplizumab, sirukumab, sofitzumab vedotin, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tarextumab, tefibazumab, telimomab aritox, tenatumomab, teneliximab, teplizumab, teprotumumab, TGN1412, ticilimumab, tigatuzumab, tildrakizumab, TNX-650, tocilizumab, toralizumab, tositumomab, tovetumab, tralokinumab, trastuzumab, TRBS07, tregalizumab, tremelimumab, tucotuzumab celmoleukin, tuvirumab, ublituximab, urelumab, urtoxazumab, ustekinumab, vantictumab, vapaliximab, varlilumab, vatelizumab, vedolizumab, veltuzumab, vepalimomab, vesencumab, volociximab, vorsetuzumab mafodotin, votumumab, zalutumumab, zanolimumab, zatuximab, ziralimumab, and zolimimab aritox.

11. A composition for extending the half-life of a crystalline antibody comprising synergistically effective amounts of lactulose and a crystalline anti-PCSK9 antibody.

12. A method of extending the half-life of a crystalline antibody, including the steps of:
administering a synergistically effective amount of a half-life-extending compound and a crystalline antibody to an individual; and
extending the half-life of the crystalline antibody.

13. The method of claim 12, wherein the half-life-extending compound is chosen from the group consisting of a non-absorbable sugar, a compound that converts NH3 to NH4+, a prebiotic, a hydrogen-generating compound, and combinations thereof.

14. The method of claim 12, wherein the half-life-extending compound is lactulose or homologues thereof and is in an amount lower than indicated for treating constipation, said amount being less than 10 g/day.

15. The method of claim 13, wherein the non-absorbable sugar is chosen from the group consisting of lactulose, glucose, galactose, fructose, mannitol, inulin, sucralose, aspartame, dextrose, maltodextrin, homologues, and combinations thereof.

16. The method of claim 13, wherein the prebiotic is chosen from the group consisting of lactulose, disaccharides, inulin, fructooligosaccharides (FOS), xylooligosaccharides (XOS), polydextrose, oligofructose-enriched inulin, resistant starch, galactooligosaccharides (GOS), transgalactooligosaccharide, mannooligosaccharides, tagatose, and monosaccharides.

17. The method of claim 13, wherein the hydrogen-generating compound is chosen from the group consisting of H₂ molecules, a prodrug releasing H₂, a compound that causes the release of H₂ within the body, an H₂ infused liquid, and combinations thereof.

18. The method of claim 13, wherein the compound that converts NH3 to NH4+ is lactulose or homologues thereof.

19. The method of claim 12, wherein the crystalline antibody is crystalline anti-PCSK9 mAb.

20. The method of claim 19, wherein the crystalline anti-PCSK9 MAb is crystalline bococizumab.

21. The method of claim 12, wherein the crystalline antibody is chosen from the group consisting of 3F8, 8H9, abagovomab, abciximad, abrilumab, actoxumab, adalimumab, adecatumumab, aducanumab, afelimomab, afutuzumab, alacizumab pegol, ALD518, alemtuzumab, alirocumab, altumomab pentetate, amatuximab, anatumomab mafenatox, anifrolumab, anrukinzumab, apolizumab, arcitumomab, aselizumab, atinumab, atlizumab, atorolumumab, bapineuzumab, basiliximab, bavituximab, bectumomab, belimumab, benralizumab, bertilimumab, besilesomab, bevacizumab, beziotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cBR96-doxorubicin immunoconjugate, CC49, cedelizumab, certolizumab pegol, cetuximab, Ch.14.18, citatuzumab bogatox, cixutumumab, clazakizumab, clenoliximab, clivatuzumab tetraxetan, cnatumumab, concizumab, CR6261, crenezumab, dacetuzumab, daclizumab, dalotuzumab, daratumumab, demcizumab, denosumab, detumomab, dinutuximab, diridavumab, dorlimomab aritox, drozitumab, duligotumab, dupilumab, durvalumab, dusigitumab, ecromeximab, eculizumab, edobacomab, edrecolomab, efalizumab, efungumab, eldelumab, elotuzumab, elsilimomab, emibetuzumab, enavatuzumab, enfortumab vedotin, enlimomab pegol, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, ficlatuzumab, figitumumab, flanvotumab, fletikumab, fontolizumab, foralumab, forvirumab, fresolimumab, fluranumab, futuximab, galiximab, ganitumab, gantenerumab, gavilimomab, gemtuzumab ozogamicin, gevokizumab, girentuximab, glembatumumab vedotin, golimumab, gomiliximab, guselkumab, ibalizumab, ibritumomab tiuxetan, icrucumab, igovomab, IMAB362, imciromab, imgatuzumab, inclacumab, indatuximab ravtansine, infliximab, inolimomab, inotuzumab ozogamicin, intetumumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, lifastuzumab vedotin, ligelizumab, lintuzumab, lirilumab, lodelcizumab, lorvotuzumab, lorvotuzumab mertansine, lucatumumab, lulizumab pegol, lumiliximab, mapatumumab, margetuximab, maslimomab, matuzumab, mavrilimumab, mepolizumab, metelimumab, milatuzumab, minretumomam, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, obiltoxaximab, ocaratuzumab, ocrelizumab, odulimomab, ofatumumab, olaratumab, olokizumab, omalizumab, onartuzumab, ontuxizumab, oportuzumab monatox, oregovomab, orticumab, otelixizumab, otlertuzumab, oxelumab, ozanezumab, ozoralizumab, pagibaximab, palivizumab, panitumumab, pankomab, panobacumab, parsatuzumab, pascolizumab, pateclizumab, patritumab, pembrolizumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samilizumab, sarilumab, satumomab pendetide, secukinumab, seribantumab, setoxaximab, sevirumab, SGN-CD19A, SGN-CD33A, sibrotuzumab, sifalimumab, siltuximab, simtuzumab, siplizumab, sirukumab, sofitzumab vedotin, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tarextumab, tefibazumab, telimomab aritox, tenatumomab, teneliximab, teplizumab, teprotumumab, TGN1412, ticilimumab, tigatuzumab, tildrakizumab, TNX-650, tocilizumab, toralizumab, tositumomab, tovetumab, tralokinumab, trastuzumab, TRBS07, tregalizumab, tremelimumab, tucotuzumab celmoleukin, tuvirumab, ublituximab, urelumab, urtoxazumab, ustekinumab, vantictumab, vapaliximab, varlilumab, vatelizumab, vedolizumab, veltuzumab, vepalimomab, vesencumab, volociximab, vorsetuzumab mafodotin, votumumab, zalutumumab, zanolimumab, zatuximab, ziralimumab, and zolimimab aritox.

22. The method of claim 12, wherein said administering step is further defined as administering the half-life-extending compound and the crystalline antibody orally.

23. The method of claim 22, further including the step of protecting the crystalline antibody from degradation until reaching the individual's gut.

24. The method of claim 12, wherein said extending step is further defined as promoting FcRn-receptor mediated pH-dependent IgG recycling process at gut epithelium.

25. The method of claim 19, wherein said extending step is further defined as dissociating the anti-PCSK9 mAb/PCSK9 immune complex during trans-epithelial trafficking of the immune complex of PCSK9-bound anti-PCSK9 mAb.

26. A method of promoting antibody recycling, including the step of:
affecting a receptor for an antibody to recycle the antibody.

27. The method of claim 26, wherein said affecting step further includes the step of creating an acidic environment in a body.

28. The method of claim 27, wherein said creating step is further defined as administering a synergistically effective amount of a half-life-extending compound and a crystalline antibody to an individual.
